(19) United States
(12) Patent Application Publication
(10) Pub. No.: US 2006/0173462 A1
(43) Pub. Date: Aug. 3, 2006

(54) Orthopedic screw for use in repairing small bones

(76) Inventors: David B. Kay, Akron, OH (US); Lee A. Strnad, Broadview Hts., OH (US); Dustin Ducharme, Akron, OH (US)

Correspondence Address: HUDAK, SHUNK & FARINE CO. LPA, Suite 307, 2020 Front Street, Cuyahoga Falls, OH, 44221, US

(21) Appl. No.: 11/340,365

(22) Filed: Jan. 26, 2006

Related U.S. Application Data
(60) Provisional application No. 60/648,209, filed on Jan. 28, 2005.

(51) Int. Cl. A61B 17/58

(52) U.S. Cl. 606/73

(57) ABSTRACT

The present invention relates to an orthopedic screw having a torque driving head with spherical surface for multiaxial use, a self starting, self tapping insertion tip and a threaded portion including a modified cancellous thread. The threaded portion has a major diameter defined by a spiraling thread and a minor diameter. The head is joined to the threaded portion by an area of from about 2 to about 6 turns of the thread along the longitudinal axis in which the minor diameter tapers by an angle of from about 4 to about 12° and the major diameter of the screw remaining substantially the same meaning that the major diameter is constant to about +/−0.05 mm along the length of the threaded portion. The screw includes a multilobe torque driving recess joined to a cylindrical recess that corresponds to a cylindrical post on the torque driver so as to form a press fit which acts to self-center the screw, to help avoid stripping of the screw/driver interface and to hold the screw in place on the torque driver prior to implantation. In a further embodiment, the screw includes a variable pitch portion for about half of the length starting from the distil end. The screw can be used be itself, or with a plate. Further a new method of using the screw with a plate is presented in which the screw extends through a cortical section of bone into a cancellous portion but not through the adjacent cortical.

This application is based on U.S. Provisional Application Ser. No. 60/648,209, filed on Jan. 28, 2005

### FIELD OF THE INVENTION

The present invention relates to an orthopedic screw for use alone and with a plate for repair or reconstruction of bones, and in particular, for use in small bones.

### BACKGROUND OF THE INVENTION

The field of orthopedic medicine has grown tremendously in the past fifty years as surgical techniques, implants and instrumentation have developed and been improved. The medical companies have tended to focus their attention on the largest market areas so that some areas of the body, such as the spine, knees and hips, have received intense focus from the large medical companies. While the small bones are frequently subject to the need for re-constructive surgery for example, as a result of trauma, to counteract the effects of aging or to repair congenital deformities, this area has typically not received the same degree of attention from the medical companies as joint replacement, trauma and spinal areas. Consequently, the products available to the small bone surgeon often represent scaled down versions of products designed for the large long bone market which are not adequate for the fine bones and delicate procedures required of the small bone surgeon. Additionally, while there is a wide variety in the exact shape and mass of all bones, these variations become more problematic in providing orthopedic implants for small bone applications since there is less room on and about the bone for the surgeon to place and fix the construct. These bones are finer and have less surface area for placement of an implant, and less mass for the placement of screws and as a result, individual variations become more problematic for implants of stock design.

One problem that needs to be avoided in the delicate environment of the small bone area is the interference of screws, with other screws, and with the function of ligaments and tendons. While it may be desirable to design an orthopedic plate and screw system so that securing screws converge in order to cause compression or increase the pullout strength, it is difficult when a screw impinges on or conflicts with the desired placement of another screw. Some surgeons prefer bicortical fixation in which a screw is sized so that the distal end is secured in cortical bone giving the screw better purchase; however, other surgeons may prefer to avoid placing a screw so that it projects beyond the outer surface of the anchoring bone. These factors are complicated by the relative lack of soft tissue and the presence of ligaments and tendons in the small bone areas. Consequently, the less forgiving biological environment in which the small bone surgeon works requires greater procedural precision and calls for specialized implants and tools.

The present invention is designed to meet the specific needs of the small bone surgeon to facilitate effective and repeatable procedures which provide for ease of use and a range of function for this specific area of specialization. The present invention could serve for the treatment of a broad range of indications including relatively straightforward fracture repair following trauma in an otherwise healthy individual where screws are used alone or with plates to maintain the integrity of the bones while they heal, as well as for more complex surgeries such as reconstruction to correct congenital or age related deformation. Reconstruction often includes arthrodesis or partial or total fusion which involves removal of a joint and the use of a mechanical-biological construct to keep the bones immobile while fusion occurs. Further small bone surgeons may be called upon to achieve soft-tissue balancing by readjusting the length of tendons and ligaments or to reshape the bone itself through removal or repositioning in a procedure known as an “osteotomy”. In an aging or diabetic population, these procedures may also involve dealing with the difficulties of poor quality bone and/or compromised soft tissue.

These surgeons typically include sub-specialists such as hand surgeons, feet and ankle and podiatric surgeons, but can also include general orthopedic surgeons who may be called upon to perform procedures on the small bones.

The present invention provides a screw for use alone or as part of a construct which could include a plate. The screw is designed specifically for the small bone market, i.e. for use in bones distil to the elbow and knee, including, for example, the ulna, radius, tibia, fibula, as well as the metacarpals, carpals, metatarsals, tarsals, and phalanges. The screw can be used in applications previously mentioned, for example those that require fixation within a single bone such as the stabilization of a fracture or the screw can be used across two or more bones so as to facilitate total or partial fusion.

The screws are self-starting, self-tapping screws including the option of partial or full cannulation. The internal recess provided by the partial or total cannulation can be used as a place to press fit a screw holder in an instrument or can be used for additional fixation, for example using a wire. The screws include a cutting end having multiple flutes, and preferably 2 or 3 flutes about a conical recess. The screws further include a cancellous thread. The screw further has a partial taper of the minor diameter of about 5° to about 15°, and more preferably about 6° to about 10°, and most preferably about 8° over about the first 2 to about 6, and more preferably about 3 or 4 complete turns of the threads. The screws are of particular advantage in that they provide for an excellent bite, and act to “suck” a plate onto the bone in instances where they used with a plate.

The screws further include a torque driving recess that may be a hexagon, a sinusoidal shape, or a modification of a sinusoidal (multilobed) shape. The recess can be of a constant size in the direction of the longitudinal axis, or can taper inward along the longitudinal axis of the screw toward the bottom of the recess. In addition, the head of the screw can include a rounded portion or spherical shaped head to permit multiaxial insertion, i.e. in a corresponding rounded or spherical recess in a countersunk screw hole in a plate or other construct. The screws can be provided in typical lengths for small bone use, i.e. from about 5 mm to about 25 mm and typically in lengths of 8, 12 16 and 20 mm with a major diameter of about 2.7 right or 3.5 mm. The screws can include a constant thread pitch as shown, in particular for use with a bone plate. A further embodiment of the screw for use in fixation by itself is a screw which includes a compression thread which increases in the number of turns over a given length. When used, this variable pitch will preferably be used for the thread over about half of the distil end of the screw. The screws can be made of appropriate biocompatible material, including for example surgical grade stainless steel and titanium.

### BRIEF DESCRIPTION OF THE DRAWINGS

FIG. 1 is a side view of an orthopedic screw in accordance with the invention;

FIG. 2 is a cross-section of the screw taken along line 2-2;

FIG. 3 is a top end view of the screw;

FIG. 4 is a bottom end view of the screw;

FIG. 5 is a top perspective view of a plate which could be used with the screw of the present invention;

FIG. 6 is a cross section taken along line 6-6 of the plate shown in FIG. 5.

### DETAILED DESCRIPTION OF THE INVENTION

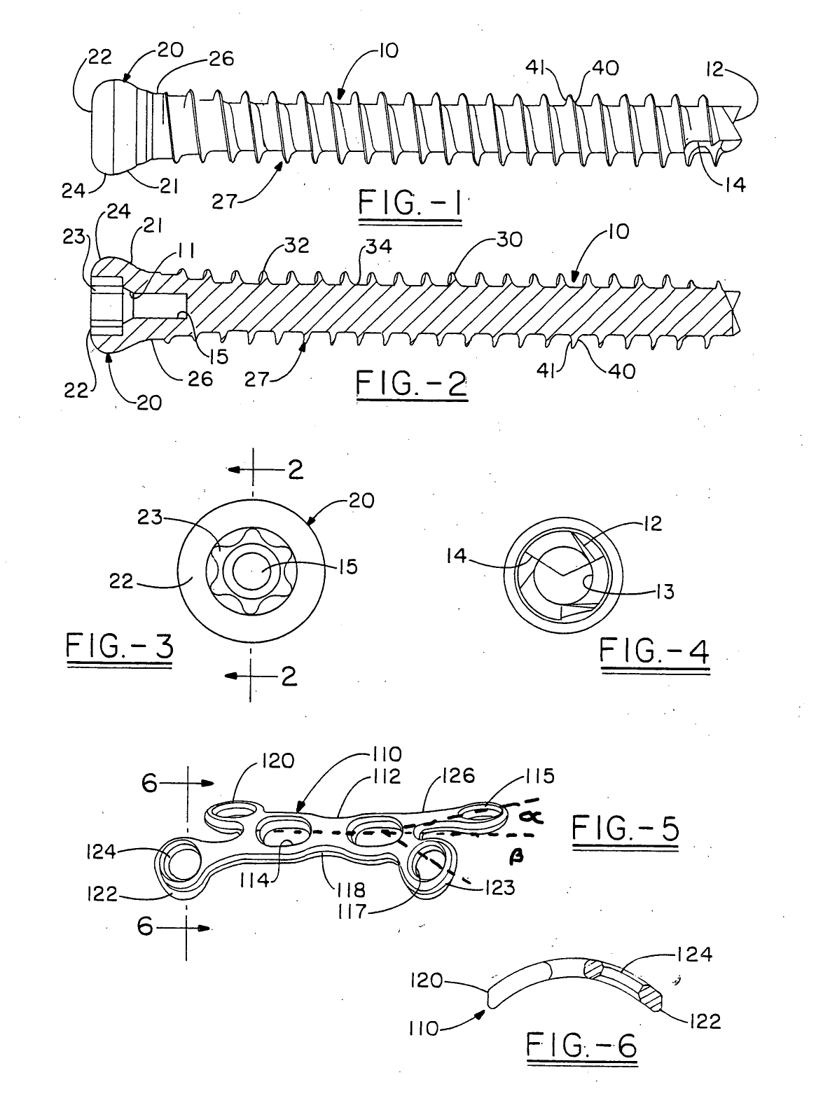

FIGS. 1-4 show an orthopedic screw 10 in accordance with the present invention. The distil end of the screw includes a cutting tip 12 which is self-starting and self-tapping. The term “distal” is used herein to mean the end that would be farthest from the point of attachment to a plate if one were used, i.e. the insertion tip, and “proximal” is used to mean the opposite end of the screw, i.e. the head. The cutting tip 12 is provided by a conical recess 13 and a plurality of flutes 14 or grooves that form sharp cutting surfaces at the terminus of the screw. The screw 10 can include a partial or full cannula 15 along its longitudinal axis. While the screw is shown as including a partial cannula in the form of a recess in the drawings, the bore can project fully toward the distal end of the screw, or can be absent. In a preferred embodiment, the screw includes the partial cannulation which is a cylindrical recess extending at least about 1.5 mm up to about 5 mm, and preferably about 2 mm to 4 mm based upon the diameter of the screw. An angled area 11 connects the cannulation or recess with the torque driving recess. The cannulation is used with a torque driving instrument that has a corresponding shaped post that will fit in the screw so that the screw is self-centering, is held in position on the torque driving instrument in a friction fit, and seats the screw so as to avoid stripping the interface between the screw and the torque driver.

The head 20 of the screw includes a rounded area 21 which preferably includes from about 0.75 mm to about 2.0 mm of a sphere having a diameter of from about 4 mm to about 5 mm. This defines a side wall which will allow for multi-axial placement in a screw hole, for example, in a plate that has a corresponding concavity. In the event that the screw is used alone, the rounded area eliminates sharp transitions between the threaded area and the head of the screw.

The screw head 20 has a relatively flat proximal surface 22 having radiused transitions 24 into the rounded area 21 of the side wall of the head. The proximal surface includes a torque driving recess 23, such as a modified multilobe shape as is shown in FIG. 3. A necked area 26 joins the rounded area 21 of the head side wall to a threaded portion 27 of the screw. The threaded portion 27 includes a cancellous thread 30 with a constant major diameter 32 which is defined by the sharp spiraling outer edge of the thread 33 which runs out to a very fine edge, (i.e. having a v-shaped return,) and a minor diameter 34 defined by the inner portion of the screw at the base of the thread. The minor diameter 34 is constant over a distal portion of the thread so as to define a cylinder with a spiraling thread. The minor diameter also includes a proximal portion that tapers inward over the length of the first four threads toward the distil end in order to improve fatigue strength and to improve compression at the proximal cortical bone interface and to compensate for bone re-adsorption. The tapered portion of the screw 36 includes a taper of from about 2° to about 20°, or more preferably from about 4° to about 12°, and most preferably about 6° to about 10° (i.e. about 8°) which tapers over from about 2 to about 10, and more preferably about 3 to about 6 complete turns (360°) of the thread 30. The pitch is between about 0.5 and 2.0 millimeters in length (i.e. a thread revolution of 360° per 0.5 to 2.0 millimeters).

The thread is a modified cancellous type thread with a front thrust 40 surface having an angle of from about 10° to about 30°, or more preferably from about 15° to about 25°, and most preferably about 18° to about 22° (i.e. about 20°) to a plane perpendicular to the longitudinal axis of the screw, while the rear surface 41 forms an angle of about 0° to about 10°, or more preferably from about 0° to about 8°, and most preferably about 3° to about 7° (i.e., about 5°) to the plane perpendicular to the longitudinal axis of the screw.

The screw can be made from an appropriate biocompatible material having appropriate strength characteristics including surgical grade stainless steel or titanium or absorptive materials.

A plate with which the screw of the present invention can be used to advantage is shown in FIGS. 5 and 6. The plate 110 is shown having a modified x-shape or asymmetrical dog-bone shape with a central trunk portion 112 defining the longitudinal axis of the plate. The trunk portion 112 includes one or preferably more elongated screw holes 114 along the longitudinal axis. The number of screw holes will depend on the length of the plate, and may range from 0 to 6. The screw holes 114 are preferably elongated to allow the plate to be set initially and subsequently to be slide into a different position and tightened down. Further, the screw holes include annular rings 115 of increased thickness in the vertical direction about through bores 117. The through bores 117 in the trunk portion have a longitudinal axis that is perpendicular to plane tangent to the top radius of the plate. The area linking the screw holes has a decreased width so as to define a waist area 118 that will bend laterally (or “curve”) relative to the longitudinal axis and which will bend longitudinally to form a curved area in and out of the plane of the plate. This thinner area also facilitates twisting of the plate so as to allow the plate to spiral, or wrap around it longitudinal axis. The increased annular area around the screw holes resists deformation when a bending device is used to apply a force to the plate through the screw holes.

The plate 110 also includes at least one set, and preferably two opposing sets of arms 120. As viewed in FIG. 5, these sets of arms can be viewed as a set of upper 122 and lower arms 123, although it is understood that the orientation of the plate can vary even after the plate has been fixed to the bone so that the terms upper and lower are only used to distinguish the pair on one end of the trunk portion 112 from the pair on the other end of the trunk portion 112. Each of the arms in a set includes screw holes 124 which are placed at a equal distance but which diverging asymmetrically from the longitudinal axis of the plate 110. More specifically, each set of arms includes a shorter arm and a longer arm and one arm that defines a smaller angle of divergence α from the longitudinal axis of the trunk portion than the angle of divergence of the other arm β. For example, the first angle shown in FIG. 5 at α may be from about 5° to about to 25°, and more preferably from about 10° to about to 20° and most preferably from about 12° to about to 16°, while the second angle shown at β from about 10° to about to 35°, and more preferably from about 15° to about to 30° and most preferably from about 22° to about to 26° with a preferred difference in the angles beings from about 2° to about to 20°, and more preferably from about 4° to about to 16° and most preferably from about 8° to about to 12°. On the inferior side, or the side that would be facing the bone surface in use, the arms continue the radius of curvature of the trunk portion. The superior or top side of the plate has a similar radius of curvature as the top surface of the plate has an outline that corresponds with the shape of the bottom of the plate (excluding the thickened annular area surrounding the screw holes.) The screw holes 124 are placed with the longitudinal axis perpendicular a tangent to the top surface of the arm with the effect that the longitudinal axes of the screws converge in the direction of the distil end. This increases the pull-out strength of the screws. Since the arms are asymmetrical relative to each other, and in particular since they diverge from the longitudinal axis of the trunk portion at differing angles, conflicts in the positions of paired screws is avoided so that the screws of a set of arms typically do not impinge on each other. This is even more important instances where the plate is bent around the longitudinal axis so as to wrap around the longitudinal axis of the bone.

The arms 120 also each include a screw hole 124 which, like the trunk portion 112 has a linking portion 126 that joins annular areas 125 of increased thickness that rings a through bore 127. Again this design facilitates the desired bending while resisting deformation of the screw holes 124 when they are used with the bending instrument to contour the plate. While the angle of the arms 120 of each one of a pair of a respective set of arms 122 and 123 varies so as to create a bilateral asymmetry, meaning that the plate is not symmetrical with respect to a plane that passes through the longitudinal axis in the vertical direction from the superior (the top side relative to the bone) to the inferior side (the side facing the bone), the “first plane”. However, the position of the arms in each set is preferably flipped so that the symmetry about a plane transverse to the first plane is a mirror image. This is defined herein as transverse mirror symmetry. Further the length of each of the arms of a pair will vary so that the transverse length from the center of the screw hole to the intersection with the longitudinal axis will be the same for the two arms. As shown in FIG. 6, the plate includes a radial curve about the longitudinal axis. The radius is typically about 10 mm with a transverse dimension from the edge of one arm to the edge of the other arm of an upper or lower pair being about 15 or 16 mm, and the screw bore having a longitudinal axis of about 24° to a plane passing through the longitudinal axis of the plate. The bores are typically about 3.75 mm for a 3.5 mm diameter screw. In a further embodiment, the bore could be threaded.

While in accordance with the patent statutes, the best mode and preferred embodiment have been set forth, the scope of the invention is not limited thereto, but rather by the scope of the attached claims.

What is claimed is:

1. An orthopedic screw comprising:
a head and a insertion tip and a threaded portion with a longitudinal axis and having a major diameter defined by a spiraling thread and a minor diameter, the head having a flat surface including a torque driving recess and joined by a bevel to a rounded side wall further joined to the threaded portion by an area of from about 2 to about 6 turns of the thread along the longitudinal axis in which the minor diameter tapers by an angle of from about 4 to about 12° and the major diameter of the screw remaining substantially the same along the length of the threaded portion, the insertion tip including a conical recess and the tip having a plurality of flutes, the screw being made from surgical stainless steel or titanium.

2. An orthopedic screw as set forth in claim 1 wherein the screw further includes at least a portion of the thread which has a variable pitch.

3. A method of stabilizing a bone segment having a surface including cortical bone superior to cancellous bone and distally opposing other cortical bone and comprising surgically accessing the bone segment, placing a plate on the surface and inserting a screw into the cortical bone and cancellous bone, but not into the distally opposing cortical bone.

4. An orthopedic screw comprising:
a head and a threaded portion having a major diameter defined by a spiraling thread and a minor diameter, the head including a torque driving recess and being joined to the threaded portion by an area in which the minor diameter tapers and the major diameter of the screw remaining substantially the same along the length of the threaded portion and the thread has at least a portion including a variable pitch.

5. An orthopedic screw as set forth in claim 4 wherein the head of the screw further includes a rounded side wall.

6. An orthopedic screw as set forth in claim 5 wherein the side wall defines at least a portion of a sphere.

7. An orthopedic screw as set forth in claim 6 wherein the screw includes a self-cutting, self tapping end.

8. An orthopedic screw as set forth in claim 4 wherein the thread is a has a front thrust surface having an angle of from about 10° to about 30° and a rear surface of from about 0° to about 10° to a plane perpendicular to the longitudinal axis of the screw and the minor diameter tapers over from about 2 to about 6 turns of the thread.

9. An orthopedic screw as set forth in claim 8 wherein the thread has a front thrust surface has an angle of from about 15° to about 25° and a rear surface of from about 0° to about 8° to a plane perpendicular to the longitudinal axis of the screw

10. An orthopedic screw as set forth in claim 9 wherein the thread has a front thrust surface has an angle of from about 18° to about 22° and a rear surface of from about 3° to about 7° to a plane perpendicular to the longitudinal axis of the screw

11. An orthopedic screw as set forth in claim 10 wherein the torque driving recess is a multilobe recess.
